(19) United States
(12) Patent Application Publication
(10) Pub. No.: US 2020/0146700 A1
(43) Pub. Date: May 14, 2020

(54) PATELLA FORMING DEVICE AND USING METHOD THEREOF

(71) Applicant: Shulan JIANG, Jiangmen City (CN)

(72) Inventor: Shulan JIANG, Jiangmen City (CN)

(21) Appl. No.: 16/188,645

(22) Filed: Nov. 13, 2018

(51) Int. Cl. A61B 17/17; A61B 17/15; A61B 17/16

(52) U.S. Cl. CPC A61B 17/1767; A61B 17/158; A61B 2017/564; A61B 2017/1602; A61B 17/1677

(57) ABSTRACT

The present invention discloses a patella forming device and a using method thereof, the patella forming device comprising a connecting member, a base, a milling member and a central shaft pin, wherein the connecting member is disposed on the top of the base, and one end of the central shaft pin is disposed on the connecting member, The other end extends vertically downwardly and extends into the hollow structure of the base, the milling member is disposed on the surface of the base, and the base is rotatable around the central shaft to remove unwanted bone tissue through the milling member; The invention rapidly rotate on the surface of the patella which needs to be formed, and removes the skeletal hypertrophy bone and the bone tissue which is not matched with the femoral side prosthesis by cutting, honing, etc., forming a smooth and regular in a short time.

### FIELD OF THE INVENTION

The invention relates to the technical field of medical instruments, in particular to a patella forming device and a using method thereof.

### BACKGROUND OF THE INVENTION

At present, in hospitals for artificial knee arthroplasty, hand-held rongeurs and other manual methods of forming patella are used. The operation time is longer, and the surface of the patella after treatment is rough. In view of the above situation, it is necessary to design a “patella forming device” dedicated to the treatment of the patella, which can effectively improve the quality of the patella after molding, reduce the operation time, reduce the patient's pain, and possibly reduce the incidence of postoperative patella pain.

### SUMMARY OF THE INVENTION

In view of the above problems, the present invention provides a patella forming device and a method for using the same, which can rapidly form a hemispherical humeral articular surface during the patella formation process of artificial knee joint replacement surgery, thereby shortening the operation time and improving the quality of surgery.

The technical solution adopted by the present invention to solve the technical problem thereof is:

The patella forming device, comprising: a connecting member for connecting with an external power device; a base with an internal hollow structure having a shape matching the patella articular surface; a milling member for cutting or milling a patella; a central shaft pin for insertion into a center of the patella for positioning; wherein the connecting member is disposed at the top of the base; One end of the central shaft pin is disposed on the connecting member, the other end of which extends vertically downwardly and extends into the hollow structure; the milling member is disposed on a surface of the base, and the base is rotatable around the central shaft pin to remove unwanted bone tissue around the patella through the milling member.

As a further improvement of the above scheme, the base is a hemispherical hollow metal body or a hemispherical hollow non-metallic hard material body.

Further, the base has a shape formed by laterally cutting a top portion of a hemispherical hollow metal body or a hemispherical hollow non-metallic hard material body.

Further, a side rasper is further disposed on the side wall of the bottom of the base.

Further, the side raspers are provided with four groups, and the four groups are evenly distributed around the bottom circumference of the base on the side walls thereof.

Further, the milling member includes a plurality of mortises, the convex portions of the plurality of mortises face inward, and the edge of the convex portion is provided with a blade.

In addition, the connecting member is a metal ring, and a cross is disposed in the metal ring; a center of the cross and a center of the base are on a longitudinal central axis of the metal ring, and the central shaft pin is placed at the center of the cross.

A method of using a patella forming device, comprising the steps of:

Step 1: First measuring the approximate diameter of the patella with a vernier scale;

Step 2: Selecting a patella forming device with a diameter slightly larger than the diameter of the patella;

Step 3: Connecting to the connecting member with the same power connection device as the common acetabulum file;

Step 4: Before shaping the patella, determining the center position of the patella and inserting the central shaft pin of the patella forming device into the center position;

Step 5: Slowly rotating the patella forming device to first remove the hyperplastic bone around the patella;

Step 6: Rotating the patella again by taking a smaller patella forming device until it forms a similar shape to the patella prosthesis, and it is better to match the femoral side prosthesis.

The present invention has the beneficial effects that: the patella forming device comprising a connecting member, a base, a milling member and a central shaft pin, and the base is rotatable around the central shaft pin to remove the bone tissue that is not needed around the patella; the invention uses a power tool to drive the patella forming device to rapidly rotate on the surface of the patella which needs to be formed, and removes the skeletal hypertrophy bone and the bone tissue that does not match the femoral side prosthesis by cutting, honing, etc. A smooth, regular surface is formed in a relatively short period of time, and the humeral surface matching the femoral side prosthesis can partially or largely replace the sacral prosthesis.

### BRIEF DESCRIPTION OF THE DRAWINGS

The present invention will be further described below in conjunction with the accompanying drawings and embodiments:

FIG. 1 is a side view of the patella forming device according to one embodiment of the present invention;

FIG. 2 is a side view of the patella forming device according to the other embodiment of the present invention;

FIG. 3 is a front elevational view of the patella forming device according to one embodiment of the present invention;

FIG. 4 is a front elevational view of the patella forming device according to the other embodiment of the present invention.

### DETAILED DESCRIPTION OF THE EMBODIMENTS

The design scheme of the present invention to achieve the above objectives is:

A patella forming device, comprising a connecting member 1 for connecting with an external power device; a base 2 having an internal hollow structure with a shape matching the patella articular surface; a milling member 3 for cutting or honing the patella; a central shaft pin 5 for insertion into the center of the patella for positioning; wherein the connecting member 1 is disposed on the top of the base 2, the central shaft pin 5 is disposed at one end on the connecting member 1, and the other end extends vertically downwardly and into the hollow structure; wherein the milling member 3 is disposed on the base; wherein the base 2 is rotatable about the central shaft pin 5 to remove unwanted bone tissue around the patella through the milling member 3. The invention adopts a power tool to drive the patella forming device to rapidly rotate on the surface of the patella which needs to be formed, and removes the skeletal hypertrophy bone and the bone tissue which is not matched with the femoral side prosthesis by cutting, honing, etc., and forms a smooth and regular surface in a short time.

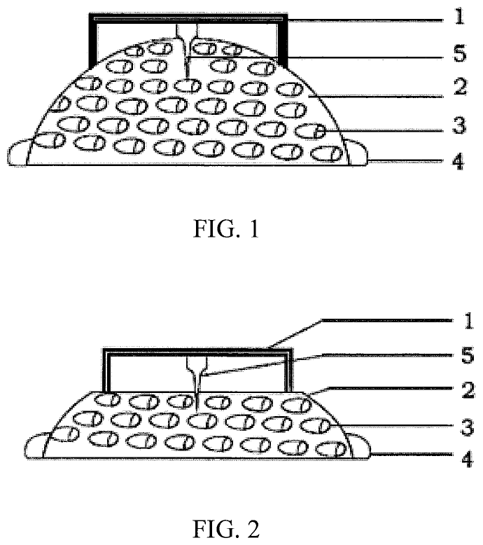

Referring to FIG. 1-4, the reference numeral 1 is a metal ring and a cross, which is mainly used for connection with a power device; the reference numeral 2 is a metal ball wall with a plurality of holes on the wall; the reference numeral 3 is a bore/hole, facing inward, and having a blade at the edge for cutting or honing the bone; the reference numeral 4 is the side rasper, for cutting or honing the bone around the bone; the reference numeral 5 is the central shaft pin, which is used to insert into the patella center for positioning and increasing the stability of rotation before the patella is cut.

In the following, a description will be given of specific embodiments by taking a plurality of types of patella molding devices as an example:

### Embodiment 1: The Patella Forming Device—Full Patella Form

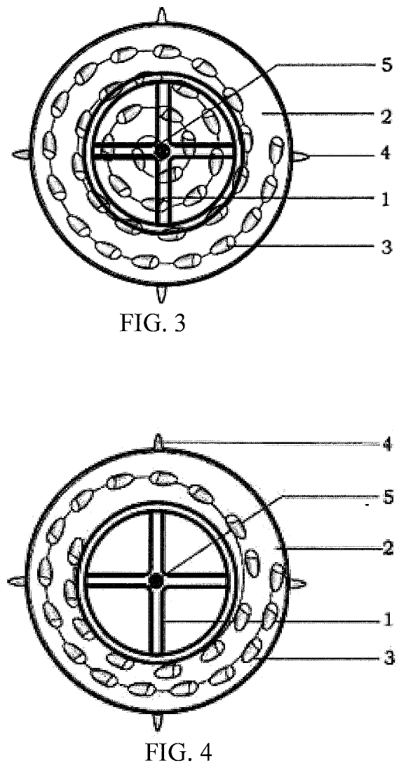

As shown in FIGS. 1 and 3, the patella forming device includes the connecting member 1 for connecting to an external power device; the base 2 having an internal hollow structure having a shape matching the patella articular surface, and the base 2 is a hemispherical hollow metal body or a hemispherical hollow non-metallic hard material body; the milling member 3 for cutting or grinding a patella; the central shaft pin 5 used for insertion into the center of the patella for positioning; the side rasper 4 disposed on a side wall of the bottom of the base 2; wherein the connecting member 1 is disposed on the top of the base 2, one end of the central shaft pin 5 is disposed on the connecting member 1, and the other end extends vertically downwardly and into the hollow structure, and the milling member 3 is disposed on the surface of the base 2, the base 2 is rotatable about the central shaft pin 5 to remove unwanted bone tissue around the patella through the milling member 3.

### Embodiment 2: The Patella Forming Device—Peripheral Patella Form

As shown in FIGS. 2 and 4, the patella forming device includes the connecting member 1 for connecting to an external power device; the base 2 having an internal hollow structure having a shape matching the patella articular surface, and the base 2 is a shape formed by laterally cutting a top portion of a hemispherical hollow metal body or a hemispherical hollow non-metallic hard material body, and the cut portion is substantially at a half position of the hemisphere; the milling member 3 used for cutting or honing the patella; the central shaft pin 5 used for inserting into the center of the patella for positioning; the side rasper 4 disposed on a side wall of the bottom of the base 2; wherein the connecting member 1 is disposed on the top of the base 2, and one end of the central shaft pin 5 is disposed on the connecting member 1, and the other end extends vertically downwardly and extends into the hollow structure, and the milling member 3 is disposed on the surface of the base 2, the base 2 is rotatable about the central shaft pin 5 to remove unwanted bone tissue around the patella through the milling member 3.

In the method for using the patella forming device of the present invention, in the artificial knee joint replacement, when the patella is formed, the diameter of the patella is first measured by the vernier scale, and the patella forming device is selected to be connected with the power device. Before shaping the patella, it is necessary to determine the center position of the patella, insert the central shaft pin of the patella forming device into the center position, and slowly rotate the patella forming device. The first step is to remove the hyperplastic bone around the patella, then take the smaller patella forming device to rotate the patella again until it is similar to the shape of the humeral prosthesis, and it is perfectly matched with the femoral side prosthesis.

The preferred embodiments of the present invention have been described in detail above. Of course, the present invention may adopt different forms and similar fields from the above-described embodiments, and these do not constitute any limitation on the present embodiment, and equivalent transformations or corresponding modifications made by those skilled in the art without departing from the spirit of the invention should fall within the scope of the invention. Of course, the above embodiments are not limitations to the invention, and other equal technical schemes should be in the protection scope of the invention.

That which is claimed is:

1. A patella forming device, comprising: a connecting member for connecting with an external power device; a base having an internal hollow structure with a shape matching the patella articular surface; a milling member for cutting or honing a patella; a central shaft pin for insertion into a center of the patella for positioning; wherein the connecting member is disposed at the top of the base; one end of the central shaft pin is disposed on the connecting member, and the other end of which extends vertically downwardly and extends into the hollow structure; wherein the milling member is disposed on a surface of the base, and the base is rotatable around the central shaft pin to remove unwanted bone tissue around the patella through the milling member.

2. The patella forming device according to claim 1, wherein the base is a hemispherical hollow metal body or a hemispherical hollow non-metallic hard material body.

3. The patella forming device according to claim 2, wherein the base has a shape formed by laterally cutting a top portion of a hemispherical hollow metal body or a hemispherical hollow non-metallic hard material body.

4. The patella forming device according to claim 3, wherein a side rasper is further disposed on the side wall of the bottom of the base.

5. The patella forming device according to claim 4, wherein the side raspers are provided with four groups, and the four groups are evenly distributed around the bottom circumference of the base on the side walls thereof

6. The patella forming device according to claim 1, wherein the milling member includes a plurality of mortises, the convex portions of the plurality of mortises face inward, and the edge of the convex portion is provided with a blade.

7. The patella forming device according to claim 2, wherein the connecting member is a metal ring, and a cross is disposed in the metal ring; a center of the cross and a center of the base are on a longitudinal central axis of the metal ring, and the central shaft pin is placed at the center of the cross.

8. A method of using a patella forming device, comprising the steps of:
Step 1: First measuring the approximate diameter of the patella with a vernier scale;
Step 2: Selecting a patella forming device with a diameter slightly larger than the diameter of the patella;
Step 3: Connecting to the connecting member with the same power connection device as the common acetabulum file;
Step 4: Before shaping the patella, determining the center position of the patella and inserting the central shaft pin of the patella forming device into the center position;
Step 5: Slowly rotating the patella forming device to first remove the hyperplastic bone around the patella;
Step 6: Rotating the patella again by taking a smaller patella forming device until it forms a similar shape to the patella prosthesis, and it is better to match the femoral side prosthesis.
